(19) United States
(12) Patent Application Publication
(10) Pub. No.: US 2026/0347740 A1
(43) Pub. Date: Nov. 5, 2026

(54) SOLID PHANTOM FOR FIXING CULTURED CELLS AND RADIATION IRRADIATION SYSTEM FOR CULTURED CELLS INCLUDING SAME

(71) Applicant: KOREA RESEARCH INSTITUTE OF STANDARDS AND SCIENCE, Daejeon (KR)

(72) Inventors: Jun-Hyuk CHOI, Daejeon (KR); In Jung KIM, Sejong (KR); Jong In PARK, Daejeon (KR); Young Min SEONG, Daejeon (KR)

(21) Appl. No.: 19/101,878

(22) PCT Filed: Apr. 30, 2024
(86) PCT No.: PCT/KR2024/005845

§ 371 Date: Feb. 6, 2025

(30) Foreign Application Priority Data
Jun. 21, 2023 (KR) 10-2023-0079764

(51) Int. Cl. C12M 1/12; C12M 1/42

(52) U.S. Cl. CPC C12M 25/00; C12M 35/00

(57) ABSTRACT

The present invention relates to a solid phantom for fixing cultured cells and a radiation irradiation system including the same, and more particularly, to a solid phantom which is easy to use and maintain by configuring a phantom block to be inserted and separated from a phantom body and capable of precisely irradiating cultured cells, and a radiation irradiation system including the same.

### TECHNICAL FIELD

The present invention relates to a solid phantom for fixing cultured cells and a radiation irradiation system including the same, and more particularly, to a solid phantom which is easy to use and maintain by configuring a phantom block to be inserted and separated from a phantom body and capable of precisely irradiating cultured cells, and a radiation irradiation system including the same.

### BACKGROUND ART

Recently, advanced medical devices that treat diseases that are difficult to surgically treat, such as head and neck cancer, congenital cerebral arteriovenous malformation, pituitary tumor, meningioma, schwannoma, metastatic brain tumor, and Parkinson's disease, through radiation have been developed and widely used.

The aforementioned advanced radiation medical devices utilize a non-invasive treatment technique that selectively and intensively irradiates a human body's affected area to remove the lesion and apply a fractionated treatment method (hereinafter, a first method) that divides a radiation exposure to normal tissue surrounding the affected area into several times at a radiation dose that normal tissue may withstand to minimize the radiation exposure.

In addition, advanced radiation medical devices apply a treatment technique (hereinafter referred to as a second method) that irradiates radiation in several directions to reduce the radiation exposure to normal tissue while neocrotizing the affected area with a large amount of radiation irradiation 1-2 times.

Currently, the first method is used in radiation therapy, and the second method is used in radiosurgery.

Meanwhile, radiation surgery has the advantage of being much less physically and mentally burdensome to patients than conventional surgical methods, has fewer side effects, such as complications, and allows patients to return to their daily lives immediately, so it is preferable despite the high cost of the procedure.

When irradiating radiation with a radiation medical device, the radiation dose absorbed by the affected area is prescribed by comprehensively considering the patient's condition, the size and characteristics of the affected area, etc., and a radiation treatment plan (RTP) is established to irradiate the appropriate dose for treatment.

Although there are differences in the sensitivity of cancer cells to radiation therapy, if a technology that may predict the sensitivity to radiation for each individual before radiation therapy is developed, cancers may be treated by adjusting the radiation dose for each individual, thereby providing the most suitable radiation therapy for each individual.

In order to determine the sensitivity of cancer cells to radiation before radiation therapy, a method of separating cancer cells, irradiating radiation thereto, and accurately and quickly analyzing the degree of death of cancer cells is required.

### RELATED ART DOCUMENT

### Patent Document

Korean Application Publication No. 10-2007-0118394 (published on Dec. 17, 2007)

### DISCLOSURE

### Technical Tasks

An object of the present invention is to provide a solid phantom which is easy to use and maintain by configuring a phantom block to be inserted and separated from a phantom body and capable of precisely irradiating cultured cells, and a radiation irradiation system including the same.

### Technical Solution

In one general aspect, a solid phantom for fixing cultured cells includes: a phantom body having an insertion recess; and a phantom block inserted and fixed into the insertion recess of the phantom body, wherein the phantom body and the phantom block are each formed of a resin material, and the phantom block includes a first phantom block into which a detector is inserted and fixed and a second phantom block into which a cell culture vessel is inserted and fixed.

The first phantom block and the second phantom block may have the same size and shape.

A horizontal beam may be irradiated to a front surface of the phantom body.

When a position in which the detector is inserted and fixed in the first phantom block is referred to as a reference position, a distance from a front surface of the second phantom block to a position in which the cell culture vessel is inserted and fixed in the second phantom block may be configured to be the same as a distance from a front surface of the first phantom block to the reference position.

The cell culture vessel may be disposed vertically with respect to the horizontal beam in the second phantom block.

One or two or more of the cell culture vessels may be stored in the second phantom block.

When a position in the second phantom block matched to the reference position in the first phantom block is referred to as a matching point, if one cell culture vessel is stored, the corresponding one cell culture vessel may be disposed on the matching point, and if two or more cell culture vessels are stored, the corresponding two or more cell culture vessels may be separately arranged to be distributed around the matching point.

The second phantom block may include a body, a vessel insertion recess formed in the body and into which the cell culture vessel is inserted, and a cover that closes the vessel insertion recess, and the cell culture vessel may be inserted into the vessel insertion recess of the body and closed by the cover.

The second phantom block may include a body, a holder fixing the cell culture vessel, and a holder insertion recess formed in the body and into which the holder is inserted, and the cell culture vessel may be fixed to the holder, and the holder to which the cell culture vessel is fixed may be inserted into the holder insertion recess of the body.

The cell culture vessel may include at least one of a flask, a plate, and a tube, and the second phantom block is provided in plurality, each of which is configured to store at least one of the flask, the plate, and the tube.

The flask, plate, and tube may each be disposed vertically with respect to the horizontal beam in the second phantom block.

The first phantom block may include a body and a detector insertion recess formed in the body and into which the detector is inserted, the detector insertion recess may be formed with an open upper portion, and the detector may be configured to be inserted and separated through the open upper portion of the detector insertion recess.

An insertion recess of the phantom body may be formed with an open upper portion, and the phantom block may be configured to be inserted and separated through the open upper portion of the insertion recess.

The phantom block may include a handle to enable the phantom block to be gripped.

The phantom block may be slidably coupled to the insertion recess of the phantom body, and an air exhaust slot may be formed on at least one side of an inner wall surface of the insertion recess and an outer wall surface of the phantom block so that air may be discharged when the phantom block is slidably coupled to the insertion recess.

Corners of a lower surface of the phantom block may be formed to be round.

The insertion recess of the phantom body may be formed in a central portion of the phantom body based on a front-rear direction of the phantom body.

The solid phantom may further include: a dummy plate formed of a resin material, wherein one or more dummy plates are disposed in a direction in which radiation is irradiated to adjust a radiation dose of radiation transmitted to the insertion recess of the phantom body.

In another general aspect, a radiation irradiation system for cultured cells includes: the solid phantom for fixing cultured cells described above; and a radiation irradiation device.

When the first phantom block is inserted into the phantom body and the radiation irradiation device irradiates radiation with a magnitude of a first energy using the detector, an absorbed dose reaching the detector may be measured, the first phantom block may be removed from the phantom body and the second phantom block is inserted into the same position, the radiation irradiation device may irradiate radiation with a magnitude identical to the magnitude of the first energy to cultured cells accommodated in a cell culture vessel of the second phantom block, and an absorbed dose reaching the cultured cells may be calculated based on the absorbed dose measured by the detector.

### Advantageous Effects

The present invention is easy to use and maintain by configuring a phantom block to be inserted and separated from a phantom body, cultured cells may be precisely irradiated with radiation.

### BRIEF DESCRIPTION OF THE DRAWINGS

FIG. 1 is a perspective view of a solid phantom according to an example of the present invention.

FIG. 2 is an exploded perspective view of the solid phantom of FIG. 1.

FIG. 3 is a perspective view of a phantom block according to an example of the present invention.

FIG. 4 is a diagram illustrating a first phantom block.

FIG. 5 is a diagram illustrating a second phantom block.

FIG. 6 is a side schematic view of FIG. 3.

FIG. 7 is a diagram illustrating a cell culture vessel according to an example of the present invention.

FIG. 8 is a diagram illustrating a second phantom block according to an example of the present invention.

FIG. 9 is an exploded perspective view of a first type of second phantom block.

FIG. 10 is an exploded perspective view of a second type of second phantom block.

FIG. 11 is an exploded perspective view of a third type of second phantom block.

FIG. 12 is a perspective view of a solid phantom according to an additional example of the present invention.

FIG. 13 is a configuration diagram of a radiation irradiation system for cultured cells according to an example of the present invention.

### DESCRIPTION OF REFERENCE NUMERALS

10: solid phantom
100: phantom body
110: insertion recess
200: phantom block
201: first phantom block
202: second phantom block
290: handle
20: detector
30: cell culture vessel
RP: reference position
MP: matching point
1000: system
90: radiation irradiation device

### Embodiments

Hereinafter, the present invention will be described with reference to the accompanying drawings.

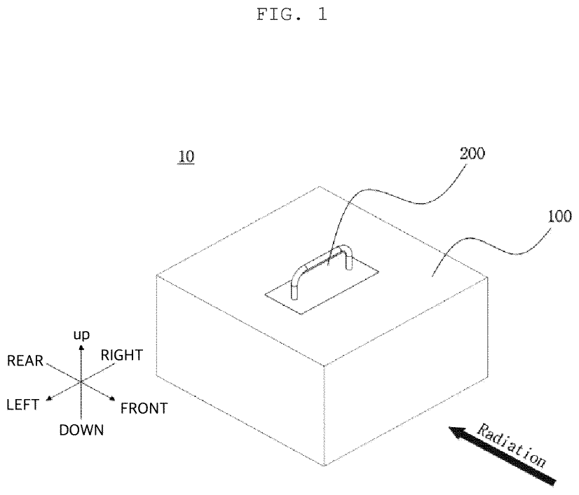

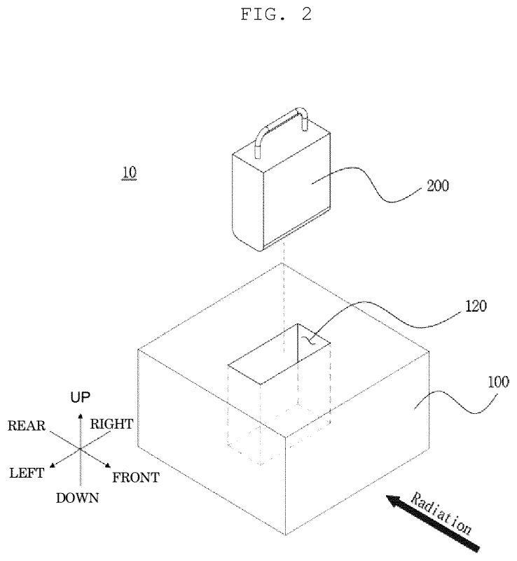

FIG. 1 is a perspective view of a solid phantom according to an example of the present invention, and FIG. 2 is an exploded perspective view of the solid phantom of FIG. 1. The solid phantom 10 of the present invention is a solid phantom for fixing cultured cells to fix cultured cells, and as illustrated, includes a phantom body 100 and a phantom block 200 assembled to the phantom body 100.

The phantom body 100 and the phantom block 200 correspond to solid phantoms each formed of a resin material. For example, the phantom body 100 and the phantom block 200 may be formed of at least one material among acrylic, polymethyl methacrylate (PMMA), and polycarbonate (PC). The phantom body 100 and the phantom block 200 may be formed of the same material, which may provide manufacturing advantages.

The phantom body 100 is configured in a roughly cubic shape, and an insertion recess 110 is formed in the phantom body 100. The insertion recess 110 of the phantom body is configured in a roughly rectangular shape that is wide in a horizontal direction.

The phantom block 200 is configured in a size and shape corresponding to the insertion recess 110 of the phantom body and is inserted and fixed in the insertion recess 110 of the phantom body.

The present invention may be set up for horizontal radiation beam irradiation. For example, a radiation irradiation device may be installed in front of the solid phantom 10 to irradiate a horizontal radiation beam (proton beam) toward the solid phantom 10, and accordingly, the corresponding horizontal beam may be irradiated to the front of the phantom body 100, pass through the phantom block 200, and be emitted to the rear of the phantom body 100. Considering the direction of the irradiation of the radiation, the phantom block 200 may be configured in a wide structure in the horizontal direction as described above.

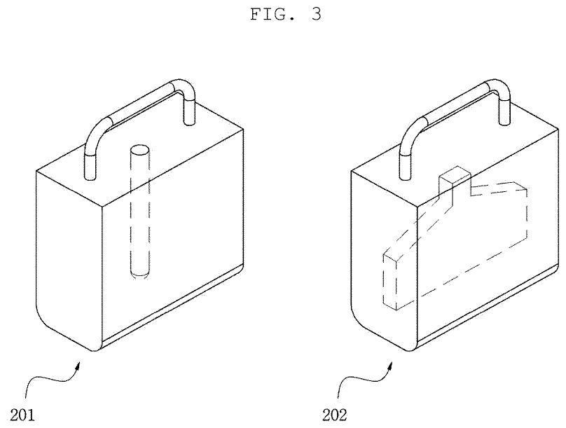

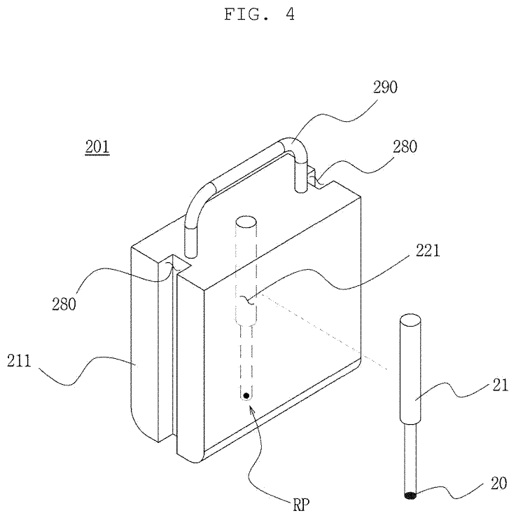

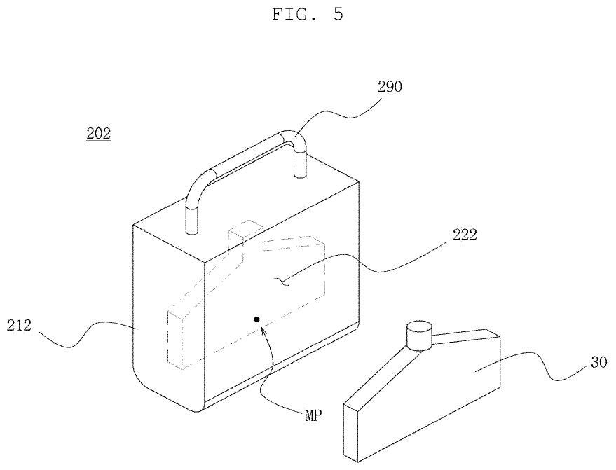

FIG. 3 is a perspective view of a phantom block according to an example of the present invention, FIG. 4 is a diagram illustrating a first phantom block, and FIG. 5 is a diagram illustrating a second phantom block. The phantom block 200 of the present invention may be classified into a first phantom block 201 in which a detector 20 is inserted and fixed and a second phantom block 202 in which a cell culture vessel 30 is inserted and fixed. That is, the present invention includes at least two phantom blocks 200.

The detector 20 is a known radiation dose measuring device that receives radiation from the outside and generates an electric signal proportional thereto and is inserted and fixed in the first phantom block 201. The cell culture vessel 30 is a known culture vessel that accommodates cultured cells and is inserted and fixed in the second phantom block 202. The first phantom block 201 and the second phantom block 202 have the same size and shape and are each inserted into the insertion recess 110 of the phantom body.

The operating principle of the present invention is as follows. First, the present invention aims to irradiate cultured cells with precise radiation, that is, to accurately determine the radiation dose irradiated to cultured cells.

To this end, the present invention separately fixes the radiation irradiation device and the phantom body 100 to preset positions, inserts the first phantom block 201 into the insertion recess 110 of the phantom body, and measures the absorbed dose at the fixed position of the detector 20 using the detector 20 inserted and fixed in the first phantom block 201. Then, the first phantom block 201 is removed from the phantom body 100, and the second phantom block 202 is inserted into the phantom body 100. At this time, since the phantom body 100 is fixed to a preset position, the fixing position of the second phantom block 202 is the same as the fixing position of the first phantom block 201, and accordingly, the radiation dose transmitted to the second phantom block 202 may be estimated by the previously measured absorbed dose. Based on this, the radiation dose irradiated to the cultured cells accommodated in the cell culture vessel 30 of the second phantom block 202 may be calculated based on the previously measured absorbed dose, thereby accurately determining the radiation dose irradiated to the cultured cells.

In this manner, according to the present invention, the dose applied to the cultured cells may be easily and simply measured while being precise, and accordingly, damage or change of the cultured cells according to the dose may be accurately analyzed.

Furthermore, since the cultured cells are generally filled with a liquid medium or a phosphate buffer solution, it is difficult to irradiate the cultured cells with a horizontal beam. A horizontal beam irradiation setup has advantages over a vertical beam irradiation setup in terms of installation and management and facility stability of the radiation irradiation device or phantom. The present invention provides a structure suitable for the horizontal beam irradiation setup for cultured cells, and thus has the advantage of allowing simple and rapid irradiation of cultured cells. Hereinafter, a specific structure of the present invention that may implement this will be described.

First, a position of the detector 20 and a position of the cell culture vessel 30 are configured to be identical to each other. More specifically, if the position in which the detector 20 is inserted and fixed in the first phantom block 201 is referred to as a reference position RP, a distance from a front surface of the second phantom block 202 to the position in which the cell culture vessel 30 is inserted and fixed in the second phantom block 202 may be configured to be identical to a distance from the front surface of the first phantom block 201 to the reference position RP.

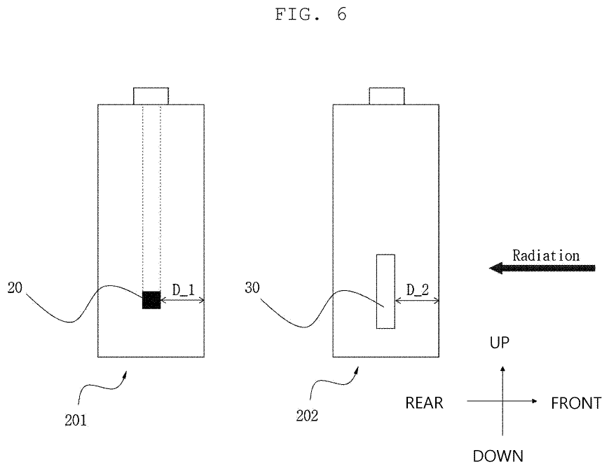

FIG. 6 is a side schematic diagram of FIG. 3, and as shown, a distance D_1 from the front surface of the first phantom block 201 to the detector 20 when the first phantom block 201 is viewed from the side and a distance D_2 from the front surface of the second phantom block 202 to the cell culture vessel 30 when the second phantom block 202 is viewed from the side may be configured to be the same. This makes a reference depth for radiation of the cultured cells match a reference depth for radiation of the detector 20, and accordingly, an error between the dose applied to the cultured cells and the absorbed dose of the detector may be reduced.

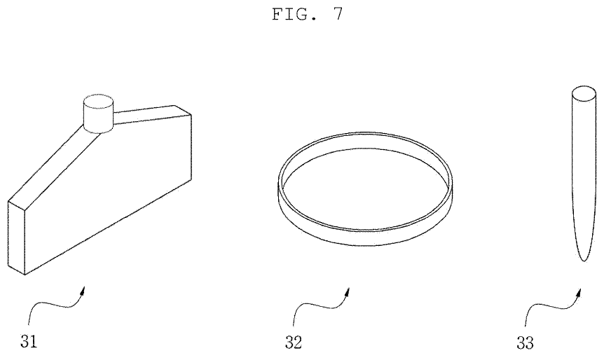

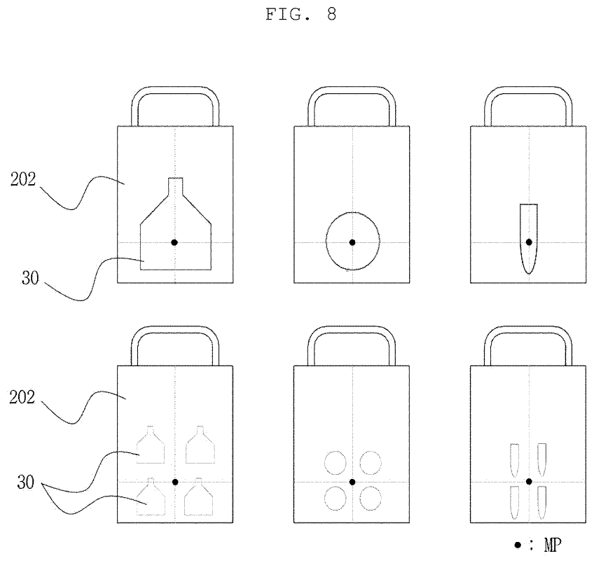

FIG. 7 is a diagram illustrating a cell culture vessel according to an example of the present invention, and FIG. 8 is a diagram illustrating a second phantom block according to an example of the present invention. As shown, the cell culture vessel 30 may be configured as various types, and based on this, the second phantom block 202 may also be configured in various forms.

More specifically, the cell culture vessel 30 includes at least one of a flask 31, a plate 32, and a tube 33, and the second phantom block 202 may be configured as a plurality of types so that each may accommodate at least one of the flask 31, the plate 32, and the tube 33.

The flask 31 is configured as a columnar body with a wide horizontal width and a constant front-rear width and has a cap that closes an open upper portion of the body, and cultured cells may be accommodated in the body. The plate 32 may be configured in a cylindrical shape and may be configured as a structure in which a cover closes the body that is open at the upper portion, and cultured cells may be accommodated between the body and the cover. The tube 33 is formed in a long pipe shape, may be open at the upper portion or may have a separate cap that closes the open upper portion, and cultured cells may be accommodated in the hollow space therein. Cultured cells may fill the internal space of the cell culture vessel.

At the same time or separately, one second phantom block 202 may be configured to accommodate one or more cell culture vessels 30. Depending on the combination of the types and number of these cell culture vessels 30, various types of second phantom blocks 202 may be manufactured. When a plurality of cell culture vessels 30 are accommodated in one second phantom block 202, it may be preferable to configure the plurality of cell culture vessels 30 to have the same type and size in order to uniformly treat the dose for each of the cultured cells accommodated in each cell culture vessel 30.

At this time, in the present invention, the cell culture vessels 30 may be installed to position corresponding to the reference position RP in order to uniformly apply the dose to the cultured cells and match the dose to the absorbed dose measured in advance.

More specifically, referring back to FIG. 8, if a position in the second phantom block 202 matched to the reference position RP in the first phantom block 201 is referred to as a matching point MP, then when one cell culture vessel 30 is accommodated, the corresponding one cell culture vessel 30 is disposed on the matching point MP, and when two or more cell culture vessels 30 are accommodated, the two or more cell culture vessels 30 may be arranged to be distributed around the matching point MP. Through this, precise and rapid radiation irradiation may be performed on a large number of cultured cells.

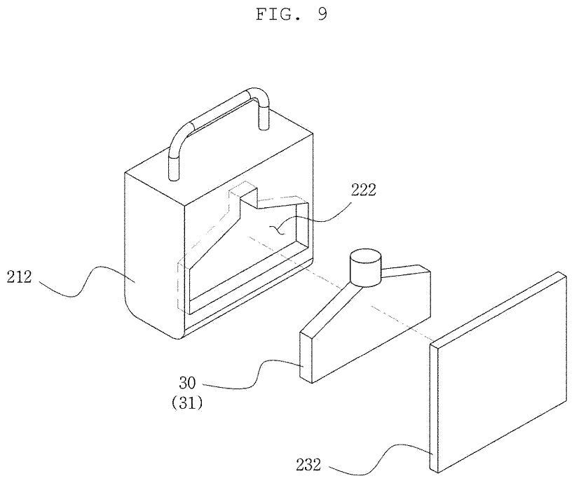

FIG. 9 is an exploded perspective view of a first type of second phantom block. As illustrated, the second phantom block 202 may include a body 212, a vessel insertion recess 222 formed in the body 212 and into which the cell culture vessel 30 is inserted, and a cover 232 that closes the vessel insertion recess 222. The cell culture vessel 30 may be inserted into the vessel insertion recess 222 of the body and may be closed by the cover 232. The body 212 and the cover 232 may be formed to have the same size and may be fixedly coupled to each other using a separate fixing unit. The cell culture vessel 30 accommodated in this type of second phantom block 202 may be the flask 31, and as illustrated, the flask 31 may be disposed vertically with respect to a horizontal beam with a wide portion thereof t disposed in a vertical direction.

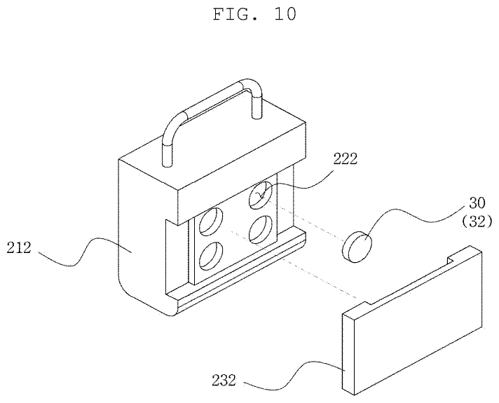

FIG. 10 is an exploded perspective view of a second type of second phantom block. As illustrated, the second phantom block 202 may include the body 212, a vessel insertion recess 222 formed in the body and into which the cell culture vessel 30 is inserted, and a cover 232 that closes the vessel insertion recess 222. The cell culture vessel 30 may be inserted into the vessel insertion recess 222 of the body and closed by the cover 232. The cover 232 may be configured to be smaller than the body in consideration of the arrangement of the cell culture vessel 30, and a recess into which the cover 232 is fitted may be formed in the body 212 so that the cover may be fitted and fixed to the body. The cell culture vessel 30 accommodated in this type of second phantom block 202 may be the plate 32, and as illustrated, the plate 32 may be disposed upright and may be disposed vertically with respect to the horizontal beam.

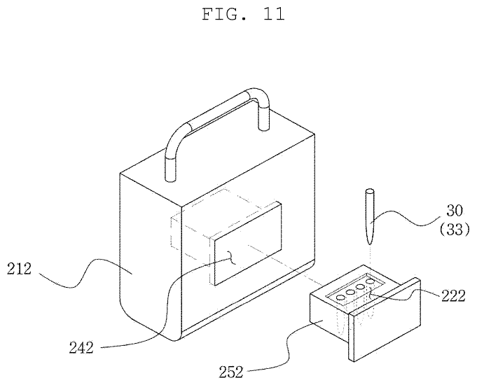

FIG. 11 is an exploded perspective view of a third type of second phantom block, and as illustrated, the second phantom block 202 may include the body 212, a holder 252 for fixing the cell culture vessel 30, and a holder insertion recess 242 formed in the body and into which the holder 252 is inserted, and the cell culture vessel 30 may be fixed to the holder 252, and the holder 252 to which the cell culture vessel 30 is fixed may be inserted into the holder insertion recess 242 of the body. The holder insertion recess 242 may be configured in a form in which both sides are open by penetrating the body or may be configured in a form in which only one side is open and the other side is closed, and the holder 252 may be fitted into the holder insertion recess 242. The cell culture vessel 30 accommodated in this type of second phantom block 202 may be a tube 33, and as illustrated, the tube 33 may be disposed vertically with a long portion thereof disposed in the vertical direction with respect to the horizontal beam.

Meanwhile, referring back to FIG. 4, the first phantom block 201 includes the body 211 and the detector insertion recess 110 formed in the body 211 and into which the detector 20 is inserted, and the detector insertion recess 221 may be formed such that the upper portion is open, and the detector 20 may be configured to be inserted and separated through the open upper portion of the detector insertion recess 221. Here, the detector insertion recess 221 may be formed with a step structure in the middle, and an arm 21 supporting the detector 20 may be configured to correspond thereto, thereby helping to o accurately position the detector 20 at the reference position RP.

Referring back to FIGS. 2 to 5, the insertion recess 110 of the phantom body may be formed with an open upper portion, and the phantom block 200 may be configured to be inserted into and separated from the open upper portion of the insertion recess 110.

In addition, the phantom block 200 may be provided with a handle 290 so that the phantom block 200 may be gripped. The handle 290 may be installed in an upper portion of the phantom block 200. Through this, a user may easily grip the phantom block 200 and easily insert and remove it from the upper portion of the phantom body 100.

In addition, the phantom block 200 may be slidably coupled to the insertion recess 110 of the phantom body. That is, the phantom block 200 may be formed to have substantially the same size as the insertion recess 110 of the phantom body and may be configured to be in close contact with an inner wall surface of the insertion recess 110 of the phantom body, which may prevent an error in the dose from occurring due to a gap formed between the phantom body 100 and the phantom block 200.

At this time, an air discharge slot may be formed on at least one side of an inner wall surface of the insertion recess 110 of the phantom body and an outer wall surface of the phantom block 200 so that air may be discharged when the phantom block 200 is slidably coupled to the insertion recess 110. The air discharge slot 280 may be formed on at least one side of the side Surfaces of the phantom block 200 as shown in FIG. 4, for example, and may be configured in a form in which the upper and lower portions extend. Through this configuration, inserting the phantom block 200 into the phantom body 100 and separating it from the phantom body 100 may be performed more easily. In addition, by forming the corner of the lower surface of the phantom block 200 to be round, the phantom block 200 may be more easily inserted into the insertion recess 110 of the phantom body.

Referring back to FIGS. 1 and 2, the phantom block 200 is disposed in the center of the front-rear direction of the phantom body 100. More specifically, the insertion recess 110 of the phantom body is formed in the center of the phantom body 100 based on the front-rear direction of the phantom body 100, and accordingly, the front portion of the phantom body 100 may be positioned in front of the insertion recess 110, and the rear portion of the phantom body 100 may be positioned in the rear of the insertion recess 110.

This may take into account even the effect of back scattering, which occurs when the radiation irradiated to the front surface of the phantom body 100 passes through the insertion recess 110 and is scattered at the rear of the phantom body 100, and thus, the dose at the reference position RP of the insertion recess 110 may be more precisely measured.

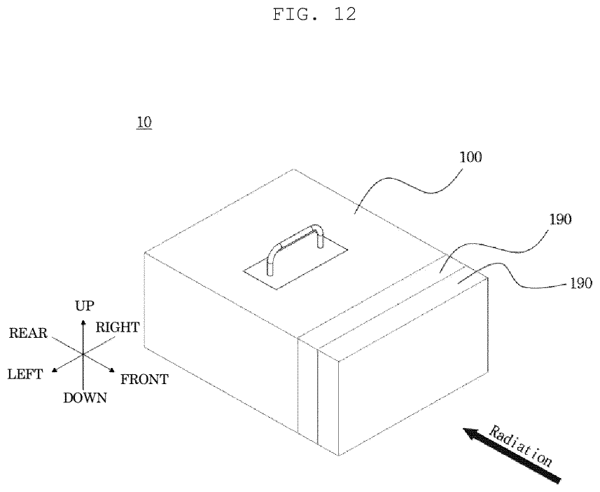

FIG. 12 is a perspective view of a solid phantom according to an additional example of the present invention, and as shown, the solid phantom 10 of the present invention may further include a dummy plate 190. The dummy plate 190 may be formed of a resin material and may be formed of the same material as the resin material of the phantom body 100.

One or more of these dummy plates 190 are mounted in the direction in which the radiation is irradiated, and accordingly, the radiation dose of the radiation transmitted to the insertion recess 110 of the phantom body may be adjusted.

For example, as illustrated, one or more dummy plates 190 may be installed in front of the phantom body 100 to adjust the reference depth to the insertion recess 110 of the radiation. The dummy plates 190 may be provided in plurality, and each of the dummy plates may have a thickness that is the same or different from each other. The dummy plates may be appropriately selected in consideration of the energy size of the radiation irradiated to the phantom body 100 and may be disposed in close contact with the phantom body 100.

In addition, although not shown, the dummy plate 190 may also be installed at the rear of the phantom body 100, which may be appropriately selected in consideration of the energy size of the radiation or an actual radiation irradiation target or environment. When the dummy plate 190 is installed at the rear of the phantom body 100, the effect of the backscattering of the radiation described above may be considered more precisely.

The dummy plate 190 may be closely fixed to the phantom body 100 through a separate bonding means, such as taping or bolting.

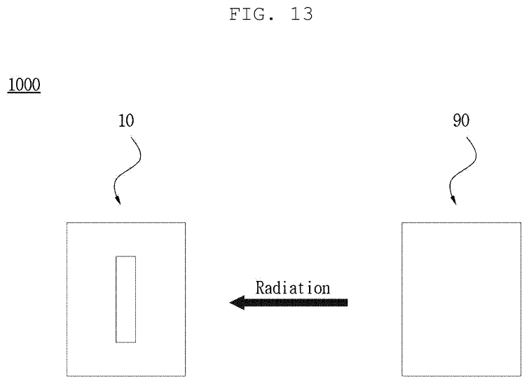

FIG. 13 is a configuration diagram of a radiation irradiation system for cultured cells according to an example of the present invention, and an irradiation system 1000 of the present invention may include the solid phantom 10 and the radiation irradiation device 90 described above, and as shown, the solid phantom 10 and the radiation irradiation device 90 may be set up in a horizontal beam. The radiation irradiation device 90 is, for example, high-energy radiation irradiation device used in a hospital for treatment and may correspond to a linear accelerator.

An operation method of the present system 1000 is as follows. First, the first phantom block 201 is inserted into the phantom body 100, and when the radiation irradiation device 90 irradiates radiation with a size of the first energy using the detector 20, the absorbed dose reaching the detector 20 is measured. Thereafter, the first phantom block 201 is removed from the phantom body 100, the second phantom block 202 is inserted into the same position, and the radiation irradiation device 90 irradiates radiation with a size equal to the size of the first energy to cultured cells accommodated in the cell culture vessel 30 of the second phantom block 202.

At this time, the absorbed dose reaching the cultured cells may be calculated based on the absorbed dose measured by the detector 20. For example, the absorbed dose reaching the cultured cells measured by the detector 20 may be used as is or partially corrected to be used.

As described above, the present invention proposes the specific structure and operation/design principle of a solid phantom for fixing cultured cells, and the radiation irradiation system for cultured cells using the solid phantom, thereby providing an advantage of being easy to use and maintain while being able to irradiate cultured cells with precise radiation.

Although the example embodiments of the present invention have been described above with reference to the accompanying drawings, those skilled in the art will understand that the present invention may be implemented in other specific forms without changing the technical idea or essential features thereof. Therefore, it should be understood that the example embodiments described above are illustrative in all aspects and do not limit the present disclosure

### INDUSTRIAL APPLICABILITY

In the present invention, the phantom block is configured to be inserted into and separated from the phantom body, and thus, the phantom block may be easily used and maintained and may irradiate cultured cells with precise radiation. Accordingly, the phantom block may be effectively utilized in the medical field for cancer cell examination, radiation therapy, etc.

1. A solid phantom for fixing cultured cells, the solid phantom comprising:
a phantom body having an insertion recess; and
a phantom block inserted and fixed into the insertion recess of the phantom body,
wherein the phantom body and the phantom block are each formed of a resin material, and
the phantom block includes a first phantom block into which a detector is inserted and fixed and a second phantom block into which a cell culture vessel is inserted and fixed.

2. The solid phantom of claim 1, wherein the first phantom block and the second phantom block have the same size and shape.

3. The solid phantom of claim 2, wherein a horizontal beam is irradiated to a front surface of the phantom body.

4. The solid phantom of claim 3, wherein, when a position in which the detector is inserted and fixed in the first phantom block is referred to as a reference position, a distance from a front surface of the second phantom block to a position in which the cell culture vessel is inserted and fixed in the second phantom block is configured to be the same as a distance from a front surface of the first phantom block to the reference position.

5. The solid phantom of claim 4, wherein the cell culture vessel is disposed vertically with respect to the horizontal beam in the second phantom block.

6. The solid phantom of claim 4, wherein one or two or more of the cell culture vessels are stored in the second phantom block.

7. The solid phantom of claim 6, wherein,
when a position in the second phantom block matched to the reference position in the first phantom block is referred to as a matching point,
if one cell culture vessel is stored, the corresponding one cell culture vessel is disposed on the matching point, and
if two or more cell culture vessels are stored, the corresponding two or more cell culture vessels are separately arranged to be distributed around the matching point.

8. The solid phantom of claim 4, wherein the second phantom block includes a body, a vessel insertion recess formed in the body and into which the cell culture vessel is inserted, and a cover that closes the vessel insertion recess, and the cell culture vessel is inserted into the vessel insertion recess of the body and closed by the cover.

9. The solid phantom of claim 4, wherein
the second phantom block includes a body, a holder fixing the cell culture vessel, and a holder insertion recess formed in the body and into which the holder is inserted, and
the cell culture vessel is fixed to the holder, and the holder to which the cell culture vessel is fixed is inserted into the holder insertion recess of the body.

10. The solid phantom of claim 4, wherein the cell culture vessel includes at least one of a flask, a plate, and a tube, and the second phantom block is provided in plurality, each of which is configured to store at least one of the flask, the plate, and the tube.

11. The solid phantom of claim 10, wherein the flask, plate, and tube are each disposed vertically with respect to the horizontal beam in the second phantom block.

12. The solid phantom of claim 4, wherein
the first phantom block includes a body and a detector insertion recess formed in the body and into which the detector is inserted,
the detector insertion recess is formed with an open upper portion, and the detector is configured to be inserted and separated through the open upper portion of the detector insertion recess.

13. The solid phantom of claim 3, wherein an insertion recess of the phantom body is formed with an open upper portion, and the phantom block is configured to be inserted and separated through the open upper portion of the insertion recess.

14. The solid phantom of claim 13, wherein the phantom block includes a handle to enable the phantom block to be gripped.

15. The solid phantom of claim 13, wherein the phantom block is slidably coupled to the insertion recess of the phantom body, and an air exhaust slot is formed on at least one side of an inner wall surface of the insertion recess and an outer wall surface of the phantom block so that air may be discharged when the phantom block is slidably coupled to the insertion recess.

16. The solid phantom of claim 15, wherein corners of lower surface of the phantom block are formed to be round.

17. The solid phantom of claim 3, wherein the insertion recess of the phantom body is formed in a central portion of the phantom body based on a front-rear direction of the phantom body.

18. The solid phantom of claim 1, further comprising:
a dummy plate formed of a resin material,
wherein one or more dummy plates are disposed in a direction in which radiation is irradiated to adjust a radiation dose of radiation transmitted to the insertion recess of the phantom body.

19. A radiation irradiation system for cultured cells, the radiation irradiation system comprising:
the solid phantom for fixing cultured cells of claim 1; and
a radiation irradiation device.

20. The radiation irradiation system of claim 19, wherein
when the first phantom block is inserted into the phantom body and the radiation irradiation device irradiates radiation with a magnitude of a first energy using the detector, an absorbed dose reaching the detector is measured,
the first phantom block is removed from the phantom body and the second phantom block is inserted into the same position,
the radiation irradiation device irradiates radiation with a magnitude identical to the magnitude of the first energy to cultured cells accommodated in a cell culture vessel of the second phantom block, and
an absorbed dose reaching the cultured cells is calculated based on the absorbed dose measured by the detector.
